(19) United States
(12) Patent Application Publication
(10) Pub. No.: US 2022/0328152 A1
(43) Pub. Date: Oct. 13, 2022

(54) INFORMATION PRESENTATION DEVICE, LEARNING DEVICE, INFORMATION PRESENTATION METHOD, LEARNING METHOD, INFORMATION PRESENTATION PROGRAM, AND LEARNING PROGRAM

(71) Applicant: NIPPON TELEGRAPH AND TELEPHONE CORPORATION, Tokyo (JP)

(72) Inventors: Masami TAKAHASHI, Tokyo (JP); Masahiro KOJIMA, Tokyo (JP); Takeshi KURASHIMA, Tokyo (JP); Tatsushi MATSUBAYASHI, Tokyo (JP); Hiroyuki TODA, Tokyo (JP)

(73) Assignee: NIPPON TELEGRAPH AND TELEPHONE CORPORATION, Tokyo (JP)

(21) Appl. No.: 17/639,892

(22) PCT Filed: Sep. 5, 2019
(86) PCT No.: PCT/JP2019/035005

§ 371 Date: Mar. 2, 2022

(51) Int. Cl. G16H 20/00; G04G 13/02

(52) U.S. Cl. CPC G16H 20/00; G04G 13/025

(57) ABSTRACT

A state acquisition unit of an information presentation device acquires a state of a user. Then, an action information acquisition unit acquires an action according to the state acquired by the state acquisition unit by inputting the state acquired by the state acquisition unit to a learning model or a learned model for outputting the action according to the state from the state of the user, the learning model or the learned model being subjected to reinforcement learning based on a reward function which outputs a reward according to the state of the user relative to a target state of the user. Then, an information output unit outputs the action acquired by the action information acquisition unit.

### TECHNICAL FIELD

The disclosed technology relates to an information presentation device, a learning device, an information presentation method, a learning method, an information presentation program, and a learning program.

### BACKGROUND ART

An increase of lifestyle-related diseases is a social problem. It is said that many lifestyle-related diseases are caused by an accumulation of unhealthy living. In prevention of the lifestyle-related diseases, it is known to be effective to intervene to promote healthy actions in a stage before a person gets sick. By intervening to take a healthy action for an object person, factors or risks of the person getting sick are reduced (for example, see Non-Patent Literature 1). However, an intervention measure such as health guidance requires expenses borne by a country or a local government and a huge burden on medical workers (for example, see Non-Patent Literature 2).

In addition, technology of notifying a user of a reminder is known (for example, see Non-Patent Literature 3).

### CITATION LIST

### Non-Patent Literature

Non-Patent Literature 1: Japan Preventive Association of Life-style related Disease, “Life-style related disease and its prevention”, http://www.seikatsusyukanbyo.com/main/yobou/01/php
Non-Patent Literature 2: Ministry of Health, Labour and Welfare, “Healthy Japan 21”, http://www.kenkounippon21.gr.jp/kenkounippon21/about/index.html
Non-Patent Literature 3: Google, “Set and manage reminders with Google Home”, https://support.google.com/googlenest/answer/7387866?co=G ENIE.Platform%3DAndroid&hl=ja

### SUMMARY OF THE INVENTION

### Technical Problem

Therefore, for example, it is conceivable to observe actions of a user such as having a meal, exercising and sleeping by using an application of a smartphone illustrated in Non-Patent Literature 3 described above or an IoT device or the like.

In this case, the action of the user is visualized and the user is notified to take a predetermined action. For example, in the case where a purpose is to improve a sleeping habit of the user, ideal bedtime for the user is set first. Then, for example, it is conceivable to issue a notification for encouraging the user to go to bed a little before the set bedtime.

However, in practice, even when the user tries to change only a certain specific action, it often does not go along with a daily life pattern. Therefore, a problem is that it is difficult for the user to act based on such notification.

For example, it is assumed that the user who usually goes to bed at 1 o'clock sets a target to go to bed by 24 o'clock in order to secure sufficient sleeping time. In this case, even if the user is notified to advance only the time to go to bed, it is difficult for the user to follow the notification when the actions usually performed before going to bed have not been finished yet.

Therefore, in order to be closer to an ideal habit without burden, it is needed to consider not only a specific action but also the entire daily actions of the user and dynamically intervene like counting backward to achieve desirable bedtime and gradually moving forward from dinner time in a previous stage.

Therefore, a problem up to now is that an action to be recommended cannot be presented in consideration of a chronological order of the actions of a user.

An object of the disclosed technology, which has been made in consideration of the point described above, is to present an action to be recommended in consideration of a chronological order of actions of a user.

### Means for Solving the Problem

A first aspect of the present disclosure is an information presentation device including: a state acquisition unit configured to acquire a state of a user; an action information acquisition unit configured to acquire an action according to the state acquired by the state acquisition unit by inputting the state acquired by the state acquisition unit to a learning model or a learned model for outputting the action according to the state from the state of the user, the learning model or the learned model being subjected to reinforcement learning based on a reward function which outputs a reward according to the state of the user relative to a target state of the user; and an information output unit configured to output the action acquired by the action information acquisition unit.

A second aspect of the present disclosure is a learning device including: a learning state acquisition unit configured to acquire a state of a user as a learning state; and a learning unit configured to acquire a learned model which outputs an action according to the state of the user by subjecting a learning model to reinforcement learning, the learning model being for outputting the action according to the state from the state of the user, based on a reward function which outputs a reward according to the learning state relative to a target state of the user, so as to increase a total sum of the reward output from the reward function.

A third aspect of the present disclosure is an information presentation method in which a computer executes processing of: acquiring a state of a user; acquiring an action according to the acquired state by inputting the acquired state to a learning model or a learned model for outputting the action according to the state from the state of the user, the learning model or the learned model being subjected to reinforcement learning based on a reward function which outputs a reward according to the state of the user relative to a target state of the user; and outputting the acquired action.

A fourth aspect of the present disclosure is a learning method in which a computer executes processing of: acquiring a state of a user as a learning state; and acquiring a learned model which outputs an action according to the state of the user by subjecting a learning model to reinforcement learning, the learning model being for outputting the action according to the state from the state of the user, based on a reward function which outputs a reward according to the learning state relative to a target state of the user, so as to increase a total sum of the reward output from the reward function.

A fourth aspect of the present disclosure is an information presentation program for making a computer execute processing of: acquiring a state of a user; acquiring an action according to the acquired state by inputting the acquired state to a learning model or a learned model for outputting the action according to the state from the state of the user, the learning model or the learned model being subjected to reinforcement learning based on a reward function which outputs a reward according to the state of the user relative to a target state of the user; and outputting the acquired action.

A fifth aspect of the present disclosure is a learning program for making a computer execute processing of: acquiring a state of a user as a learning state; and acquiring a learned model which outputs an action according to the state of the user by subjecting a learning model to reinforcement learning, the learning model being for outputting the action according to the state from the state of the user, based on a reward function which outputs a reward according to the learning state relative to a target state of the user, so as to increase a total sum of the reward output from the reward function.

### Effects of the Invention

According to the disclosed technology, an action to be recommended can be presented in consideration of a chronological order of actions of a user.

### BRIEF DESCRIPTION OF DRAWINGS

FIG. 1 is an explanatory drawing for explaining an outline of a present embodiment.

FIG. 2 is a block diagram illustrating hardware configurations of an information presentation device 10 of the present embodiment.

FIG. 3 is a block diagram illustrating hardware configurations of a learning device 20 of the present embodiment.

FIG. 4 is a block diagram illustrating an example of functional configurations of the information presentation device 10 and the learning device 20 of the present embodiment.

FIG. 5 is an explanatory drawing for explaining an interaction between an agent corresponding to a learned model and a user of the embodiment.

FIG. 6 is an explanatory drawing for explaining intervention by an agent corresponding to a learned model.

FIG. 7 is a flowchart illustrating a flow of information presentation processing by the information presentation device 10.

FIG. 8 is a flowchart illustrating a flow of learning processing by the learning device 20.

### DESCRIPTION OF EMBODIMENTS

Hereinafter, an example of the embodiment of the disclosed technology will be explained with reference to the drawings. Note that the same reference signs are imparted to the same or equivalent components and parts on respective drawings. In addition, a dimension ratio of the drawings is exaggerated for convenience of explanation and may be different from an actual ratio.

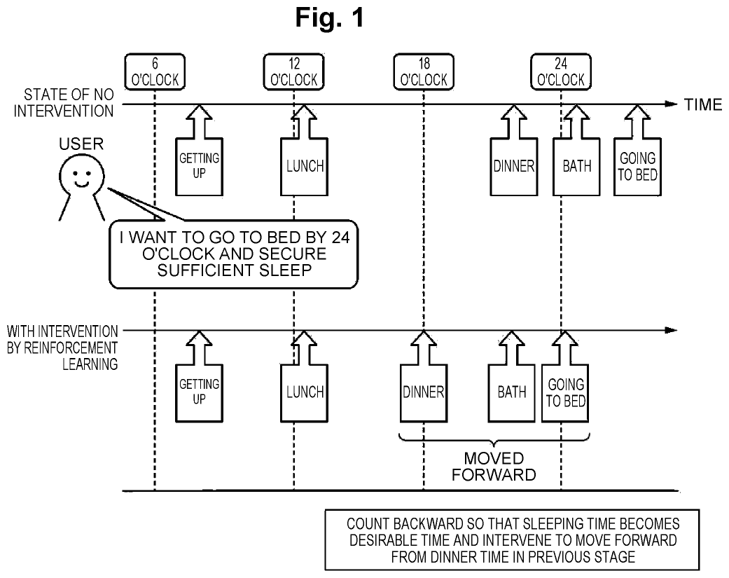

The present embodiment appropriately presents information relating to an action to a user so as to be a state targeted by the user. For example, FIG. 1 illustrates a case where a user who goes to bed at 1 o'clock everyday sets a target to go to bed by 24 o'clock in order to secure sufficient sleeping time.

In this case, it is assumed that information is presented to the user so as to advance only the time to go to bed. However, as illustrated in FIG. 1, even when the user receives presentation of such information, it is difficult to take an action according to the presented information when the actions usually performed before going to bed have not been finished yet.

Therefore, in order to bring the state of the user closer to an ideal habit without burden, it is needed to count backward to achieve desirable bedtime and present information from the actions in a previous stage. For example, like gradually moving forward from dinner time, it is needed to take not only a specific action but also the entire daily actions into consideration and dynamically intervene.

A problem with a conventional system is that the system just presents only the action to be improved, and the action cannot be dynamically presented in consideration of the entire daily actions of the user.

Then, in the present embodiment, in order to bring daily different schedules closer to an ideal lifestyle habit, the actions other than the action to be improved are also taken into consideration and prospective intervention is performed. Specifically, a learning model to be subjected to learning by reinforcement learning or a learned model which has been already subjected to the reinforcement learning is used, and the actions in the previous stage are presented such that the bedtime of the user becomes the desirable time, for example. In an example illustrated in FIG. 1, the recommended actions are presented to the user so as to move the actions of “dinner” and “bath” forward, for example. Thus, the state of the user comes closer to the target and the bedtime of the user can be brought closer to 24 o'clock.

Hereinafter, specific explanation will be given.

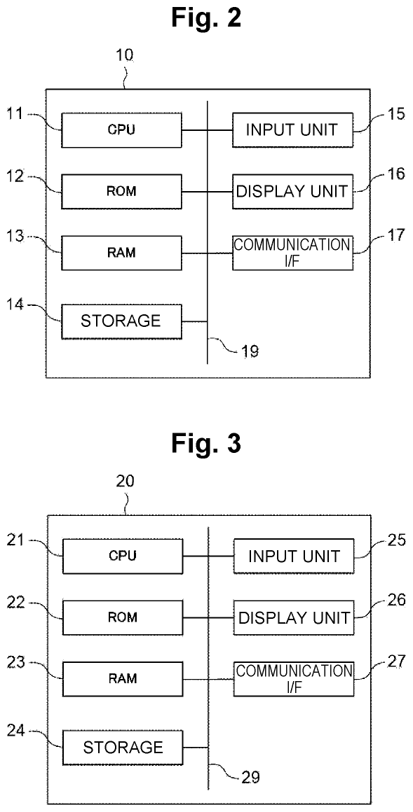

FIG. 2 is a block diagram illustrating hardware configurations of an information presentation device 10 of the embodiment.

As illustrated in FIG. 2, the information presentation device 10 of the embodiment includes a CPU (Central Processing Unit) 11, a ROM (Read Only Memory) 12, a RAM (Random Access Memory) 13, a storage 14, an input unit 15, a display unit 16, and a communication interface (I/F) 17. Each configuration is mutually communicably connected via a bus 19.

The CPU 11 is a central processing unit, executes various kinds of programs and controls the individual units. That is, the CPU 11 reads the program from the ROM 12 or the storage 14, and executes the program with the RAM 13 as a work area. The CPU 11 performs control of each configuration described above and various kinds of arithmetic processing according to the program stored in the ROM 12 or the storage 14. In the present embodiment, in the ROM 12 or the storage 14, the various kinds of programs which process information input from an input device are stored.

The ROM 12 stores the various kinds of programs and various kinds of data. The RAM 13 temporarily stores the program or the data as the work area. The storage 14 is made up of an HDD (Hard Disk Drive) or an SSD (Solid State Drive) or the like, and stores the various kinds of programs including an operating system, and the various kinds of data.

The input unit 15 includes a pointing device such as a mouse, and a keyboard, and is used to perform various kinds of input.

The display unit 16 is a liquid crystal display for example, and displays various kinds of information. The display unit 16 may function as the input unit 15 by adopting a touch panel system.

The communication I/F 17 is an interface for communicating with other equipment such as an input device, and for example, a standard such as Ethernet®, FDDI or Wi-Fi® is used.

FIG. 3 is a block diagram illustrating hardware configurations of a learning device 20 of the embodiment.

As illustrated in FIG. 3, the learning device 20 of the embodiment includes a CPU 21, a ROM 22, a RAM 23, a storage 24, an input unit 25, a display unit 26, and a communication I/F 27. Each configuration is mutually communicably connected via a bus 29.

The CPU 21 is a central processing unit, executes various kinds of programs and controls the individual units. That is, the CPU 21 reads the program from the ROM 22 or the storage 24, and executes the program with the RAM 23 as a work area. The CPU 21 performs control of each configuration described above and various kinds of arithmetic processing according to the program stored in the ROM 22 or the storage 24. In the present embodiment, in the ROM 22 or the storage 24, the various kinds of programs which process information input from the input device are stored.

The ROM 22 stores the various kinds of programs and various kinds of data. The RAM 23 temporarily stores the program or the data as the work area. The storage 24 is made up of an HDD or an SSD, and stores the various kinds of programs including the operating system, and the various kinds of data.

The input unit 25 includes a pointing device such as a mouse, and a keyboard, and is used to perform various kinds of input.

The display unit 26 is a liquid crystal display for example, and displays various kinds of information. The display unit 26 may function as the input unit 25 by adopting the touch panel system.

The communication I/F 27 is an interface for communicating with other equipment such as an input device, and for example, the standard such as Ethernet®, FDDI or Wi-Fi® is used.

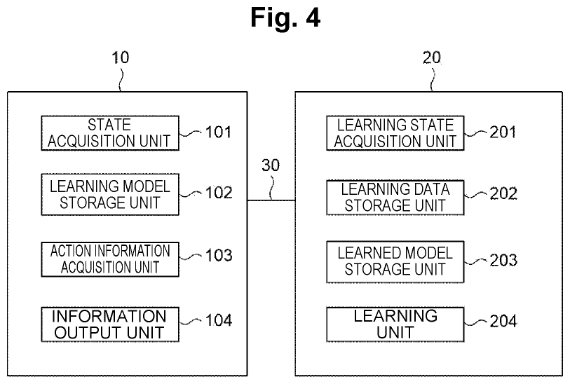

Next, functional configurations of the information presentation device 10 and the learning device 20 will be explained. FIG. 4 is a block diagram illustrating an example of the functional configurations of the information presentation device 10 and the learning device 20. The information presentation device 10 and the learning device 20 are connected by predetermined communication means 30.

[Information Presentation Device 10]

As illustrated in FIG. 4, the information presentation device 10 includes a state acquisition unit 101, a learning model storage unit 102, an action information acquisition unit 103, and an information output unit 104, as the functional configurations. Each functional configuration is implemented by the CPU 11 reading an information presentation program stored in the ROM 12 or the storage 14, loading the program in the RAM 13 and executing the program.

The state acquisition unit 101 acquires a state of a user at current time.

Note that the case where the state acquisition unit 101 of the present embodiment acquires information indicating the user and information indicating an environment in which the user is placed as the state of the user will be explained as an example.

As one example of the information indicating the environment in which the user is placed, the state acquisition unit 101 acquires observable information such as the time, a place or weather. In addition, as one example of the information indicating the user, the state acquisition unit 101 acquires observable information such as an action of the user or a health state of the user. Note that the state acquisition unit 101 executes analysis processing so as to convert the acquired information indicating the state of the user to a processable format.

Specifically, for example, the state acquisition unit 101 acquires the information acquired by an application of a smartphone carried by the user or a wearable device worn by the user or the like as the state of the user.

Or, for example, the state acquisition unit 101 may acquire the information for which the action of the user is input in a format of text or the like as a lifelog as the state of the user. Or, for example, the state acquisition unit 101 may acquire the state of the user from a schedule of the user or the like. Since the state of the user can be observed and acquired by existing technology, the information indicating the state is not limited in particular, and can be implemented in various forms.

The state acquisition unit 101 outputs the acquired state of the user to the action information acquisition unit 103. In addition, the state acquisition unit 101 transmits the acquired state of the user to the learning device 20 via the communication means 30.

In the learning model storage unit 102, a learning model scheduled to be subjected to the learning by the learning device 20 or a learned model which has been already subjected to the reinforcement learning is stored. The learning model is a model subjected to the reinforcement learning based on a reward function which outputs a reward according to the state of the user at the current time relative to a target state of the user in the future (for example, see reference literature (Reinforcement learning: An introduction, Richard S Sutton and Andrew G Barto, MIT press Cambridge, 1998)). In addition, the learned model is a model which has been already subjected to the learning by the reinforcement learning.

The information presentation device 10 of the present embodiment uses the learning model or the learned model to determine what kind of intervention is to be performed to the user in order to bring the state of the user closer to the ideal lifestyle habit. The learned model is subjected to the learning by the learning device 20 to be described later. A specific generation method of the learned model will be described later.

The action information acquisition unit 103 acquires the action according to the current state of the user by inputting the current state of the user acquired by the state acquisition unit 101 to the learning model or the learned model stored in the learning model storage unit 102. The information indicating the action indicates the intervention to the current state of the user. Note that, at the time of acquiring the action according to the current state of the user for the first time, in a situation where data is not obtained yet, the action information acquisition unit 103 uses the learning model stored in the learning model storage unit 102 to acquire the action according to the current state of the user. In addition, when the action information acquisition unit 103 acquires the action according to the state of the user for the second time and thereafter, it is the situation where the data is obtained, and the learned model subjected to the reinforcement learning by the learning device 20 to be described later has been obtained. Therefore, the action information acquisition unit 103 uses the learned model stored in the learning model storage unit 102 to acquire the action according to the current state of the user.

The information output unit 104 outputs the action acquired by the action information acquisition unit 103. Thus, the user takes the next action according to the information indicating the action, which is output from the information output unit 104.

The learned model stored in the learning model storage unit 102 has been subjected to the learning beforehand by the learning device 20 to be described later. Therefore, from the learned model, an appropriate action for the current state of the user is presented.

[Learning Device 20]

As illustrated in FIG. 4, the learning device 20 includes a learning state acquisition unit 201, a learning data storage unit 202, a learned model storage unit 203, and a learning unit 204 as the functional configurations. Each functional configuration is implemented by the CPU 21 reading a learning program stored in the ROM 22 or the storage 24, loading the program in the RAM 23 and executing the program.

The learning state acquisition unit 201 acquires the state of the user transmitted from the state acquisition unit 101 as a learning state. Then, the learning state acquisition unit 201 stores the acquired learning state in the learning data storage unit 202.

In the learning data storage unit 202, a plurality of learning states are stored. For example, in the learning data storage unit 202, the learning states at the respective times of the user are stored. The learning states stored in the learning data storage unit 202 are used for the learning of the learned model to be described later.

In the learned model storage unit 203, the learning model for outputting the action according to the state from the state of the user is stored. A parameter included in the learning model is learned by the learning unit 204 to be described later. Note that the learning model of the present embodiment may be any model as long as it is a known model.

The learning unit 204 subjects the learning model stored in the learned model storage unit 203 to the reinforcement learning, and generates the learned model for outputting the action according to the state from the state of the user. Note that, in the case where the learned model is already stored in the learned model storage unit 203, the learning unit 204 updates the learned model by subjecting the learned model to the reinforcement learning again.

The reinforcement learning used in the learning unit 204 is a method in which an agent (a robot or the like for example) corresponding to the learning model estimates an optimum action rule (also referred to as “measure”) through an interaction with the environment.

The agent corresponding to the learning model observes the environment including the state of the user, and selects a certain action. Then, by execution of the selected action, the environment including the state of the user is changed.

In this case, to the agent corresponding to the learning model, some reward is given accompanying the change of the environment. At the time, the agent learns selection of the action so as to maximize a cumulative sum of the rewards in the future.

In the reinforcement learning relating to the present embodiment, “environment” in the reinforcement learning is set as the user himself/herself, and “state” in the reinforcement learning is set as the state of the user (for example, when and what the user is doing, or the like). In addition, “action” in the reinforcement learning is set as the intervention to approach the user. Then, to the learning model corresponding to the agent, a positive or negative reward is given according to whether or not the user lives along the target state as the target. The learning model corresponding to the agent learns an intervention measure indicating the action by trial and error so as to get closer to the ideal lifestyle habit indicated by the target state of the user.

Note that the reward function of the present embodiment outputs the reward according to the state of the user at the current time relative to the target state of the user in the future. Specifically, the reward function is a function which outputs a larger reward as the state of the user at the current time comes closer to the target state of the user in the future. Further, the reward function is a function which outputs a smaller reward as the state of the user at the current time separates farther from the target state of the user in the future.

Therefore, the reward function outputs the reward according to an achievement level of the target state of the user. The reward output from the reward function is obtained according to an ideal habit or a healthy action. Note that the target state of the user is digitized in some form and set.

In the present embodiment, “environment” in the reinforcement learning is set as the user himself/herself, however, in the case where “environment” in the reinforcement learning is a simulator of the user, the state of the user can be simulated by a method of modeling and predicting the state of the user from a past history or the like. Therefore, the agent corresponding to the learning model can also perform the learning based on the state of the user obtained by the simulator of the user.

In the reinforcement learning, as a setting of “environment”, a Markov decision process (MDP) is utilized in many cases. Therefore, the Markov decision process is utilized also in the present embodiment.

The Markov decision process describes the interaction between the agent corresponding to the learning model and the environment and is defined by four-piece set information (S, A, P_{M}, R).

Here, S is referred to as a state space, and A is referred to as an action space. In addition, s∈S is the state and a∈A is the action. The state space S indicates a set of the states that the user can take. Further, the action space A is a set of the actions that can be taken to the user.

PM:S×A×S→[0,1] is referred to as a state transition function, and is a function which determines a transition probability to a next state s′ when the user receives recommendation of an action “a” indicating the intervention in a certain state s.

A reward function R:S×A×S→R defines goodness of the action “a” recommended to the user in the certain state s as a reward. The agent corresponding to the learning model selects the action “a” indicating the intervention so as to increase the sum of the rewards obtained in the future in the setting described above as much as possible. A function which determines the action “a” to be executed when the user is in the individual state s is referred to as a measure, and is described as π:S×A→[0,1].

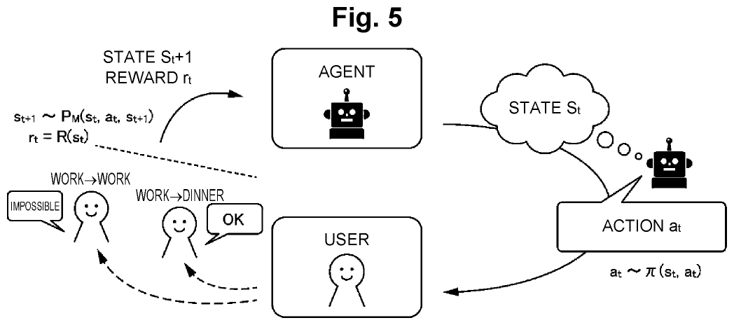

Here, when one measure is determined, the agent corresponding to the learning model becomes possible to interact with the environment as illustrated in FIG. 5. The user takes some state s∈S at all times, and the agent in a state sₜ at each time t determines an action aₜ indicating the intervention according to a measure π(·|st). At the time, according to the state transition function and the reward function, a state sₜ₊₁˜P_{M}(·|sₜ, aₜ) and a reward rₜ=R(sₜ, aₜ) at the next time of the agent corresponding to the learning model are determined. By repetition of the determination of the action according to the measure and the determination of the state and the reward at the next time, a history of the state s and the action “a” indicating the intervention is obtained.

Hereinafter, the history (s₀, a₀, s₁, a₀, . . . , s_{T}) of the state repeatedly changed for T times from a time 0 and the action indicating the intervention is indicated as d_{T}. In addition, hereinafter, d_{T} is referred to as an episode.

Here, a function which is referred to as a value function and has a role of indicating the goodness of the measure is defined. The value function is defined as an average of the sums of discounted rewards when the action “a” indicating the intervention is selected in the state s and the intervention is continuously performed according to the measure after the action “a” is selected, and is indicated by the following expression.

$$[ Math . 1 ]$$

$${Q}^{π} ( s , a ) = {lim}_{T \to ∝} {{d}_{T}}_{π}^{} [ {\sum }_{k = 0}^{T} {γ}^{k} R ( {s}_{k} , {a}_{k} , {s}_{k} + 1 ) \vert {s}_{0} = s , {a}_{0} = a ]$$

Provided that γ∈[0,1) indicates a discount rate. In addition, a sign indicated in the following expression indicates an average operation regarding output of the episode by the measure π.

[symbol]

_{d}_{_{T}}^{π[ ]} [Math. 2]

It is assumed that certain measures π and π′ satisfy the following expression in arbitrary s∈S and a∈A.

Q^{π}(s,a)≥Q^{π′} [Math. 3]

In this case, the measure π is expected to bring a larger reward than the measure π′, it is indicated as the following expression.

π≥π′ [Math. 4]

An optimum measure is obtained by setting an expression as the following expression using an optimum value function Q *.

π*(a|s)=δ(a−arg max_{a′}Q*(s,a′)) [Math. 5]

It is known that the optimum value function satisfies an optimum Bellman equation indicated in the following expression (1). Therefore, by using a relational expression of the following expression (1), the action “a” to be presented is selected or estimated.

$$\begin{matrix}[ Math . 6 ] & \\ {Q}^{⋆} ( s , a ) = {{s}^{′}}_{} [ R ( s , a , {s}^{′} ) + γ {max}_{{a}^{′}} {Q}^{⋆} ( {s}^{′} , {a}^{′} ) ] & ( 1 )\end{matrix}$$

Note that the learning unit 204 of the present embodiment performs the reinforcement learning using Q learning (for example, see reference literature (Christopher J C H Watkins and Peter Dayan, “Q-learning. Machine learning, Vol. 8, No. 3-4, pp. 279-292, 1992)), and generates the learned model which outputs the action “a” according to the state s of the user. Further, the learning unit 204 of the present embodiment is described with the case of generating the learned model using the Q learning as an example, but may generate the learned model using other methods.

When the learned model is generated by the learning device 20, the learned model in the learned model storage unit 203 of the learning device 20 is updated. In addition, the learned model stored in the learned model storage unit 203 of the learning device 20 is transmitted to the information presentation device 10 and stored in the learning model storage unit 102.

Then, the action information acquisition unit 103 of the information presentation device 10 inputs the state s acquired by the state acquisition unit 101 to the learned model stored in the learning model storage unit 102, and acquires the action “a” output from the learned model. Further, the action information acquisition unit 103 may output the action “a” to be presented to the user after narrowing down action candidates output from the learned model. The action “a” is the information indicating an approach for urging the healthy action to the user. Then, the information output unit 104 of the information presentation device 10 displays the action “a” output from the learned model at the display unit 16.

The user confirms the action “a” displayed at the display unit 16. Then, for example, the user takes an actual action corresponding to the action “a”. When a predetermined action is taken by the user, as a result, the state of the user becomes a new state.

When the new state of the user is acquired, the state acquisition unit 101 of the information presentation device 10 transmits the new state of the user to the learning device 20. The learning state acquisition unit 201 of the learning device 20 acquires the new state of the user transmitted from the information presentation device 10, and stores the new state in the learning data storage unit 202. In this case, in learning processing in the learning unit 204, the reward according to the new state of the user is obtained.

When presenting the action “a” output from the information presentation device 10, various kinds of means, contents and timing or the like are selectable. For example, the information presentation device 10 is implemented by a smartphone carried by the user or a wearable device worn by the user. In this case, for example, a message indicating the action “a” is displayed at the display unit 16 of the terminals. Or, in the case where the terminals have a vibrating function, the information indicating the action “a” is presented by a vibration signal.

Or, the information presentation device 10 may present the information indicating the action “a” to the user by utilizing a device existing around the user such as a robot or a smart speaker. Other than that, various methods of presenting the action “a” such that the user directly or indirectly changes the action, and urging the user to take a predetermined action may be adopted.

In addition, in the case where “it is desirable to take an action of having dinner at the certain time” is selected as content of specific presentation of the action “a”, the information presentation device 10 presents “dinner” indicating the action “a” at the certain time as it is. Or, the information presentation device 10 may generate some message such as “How about having dinner?” or “Let's have dinner three hours before going to bed” as the information indicating the action “a”, and present the information indicating the action “a”.

Further, the information presentation device 10 may generate a specific vibration indicating the action “a” or a light pattern indicating the action “a” and notify the user of the content of the action “a”. In addition, the information presentation device 10 may not only indicate the time, a day of the week, a month and a year or the like for timing of presenting the action “a” as intervention but also add a condition such as “after the user takes a certain action” or “when an activity amount of the user exceeds a certain threshold”, and present the information indicating the action “a”.

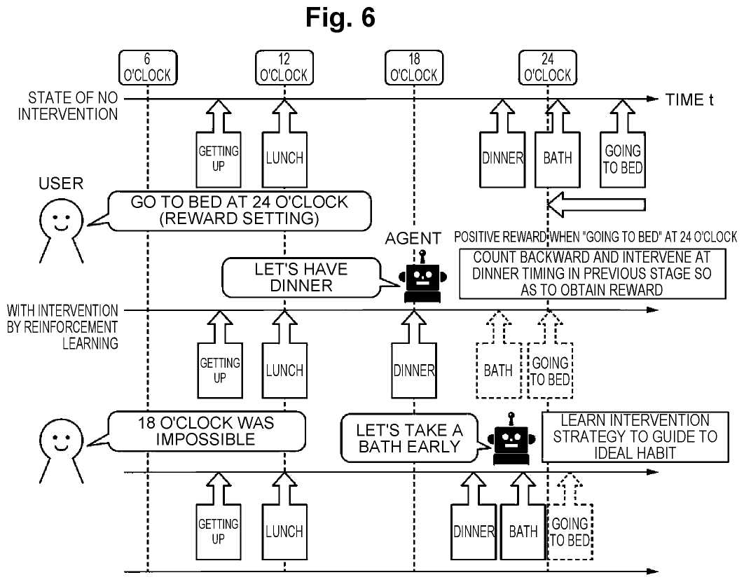

FIG. 6 illustrates an operation example of the present embodiment. FIG. 6 is an example of the case where it is ideal for the user to go to bed at 24 o'clock and the target state of the user is set as “go to bed at 24 o'clock”. By setting the target state of the user is set as “go to bed at 24 o'clock”, sleeping time of the user is sufficiently secured and the lifestyle habit is improved. The example in FIG. 6 is the example of learning the measure of presenting the action “a” indicating the intervention and bringing the action of the user closer to the ideal habit.

In FIG. 6, the state s of the user is the time indicated in a 24-hour unit and the action taken by the user. The state acquisition unit 101 of the information presentation device 10 acquires the state of the user such as “9:00 getting up”, “12:00 lunch”, “21:00 dinner” and “24:00 bath” as input. Then, the state acquisition unit 101 outputs the acquired state of the user to the action information acquisition unit 103. At the time, when the state of the user is not in a format processable in the individual units of the individual devices, the state acquisition unit 101 performs the analysis processing or conversion processing to the state of the user, and converts the state of the user to the processable format. In addition, the state acquisition unit 101 transmits the state of the user to the learning device 20. The learning state acquisition unit 201 of the learning device 20 acquires the state of the user transmitted from the information presentation device 10 as the learning state, and stores the learning state in the learning data storage unit 202.

For example, the information presentation device 10 is implemented by a robot. It is assumed that timing of presenting the action “a” by the information presentation device 10 is at an interval of one hour after the user gets up until the user goes to bed, and the content is selected from the actions that the user can take and recommended. Then, the information presentation device 10 notifies the user of the message such as “Let's eat dinner” or “Let's take a bath early” through the robot.

In this case, since the target state of the user is “going to bed at 24 o'clock”, the reward function R is defined as the function which gives a larger positive reward as “going to bed” of the user is performed at the time closer to 24 o'clock. In addition, the reward function R is defined as the function which gives the negative reward as “going to bed” of the user is performed at the time later than 24 o'clock.

Further, the information regarding initial settings such as the fact that one day is 24 hours, the means, the timing and the content for presenting the action “a”, the information indicating the configured Markov decision process and the discount rate for the reward is stored in a predetermined storage unit beforehand. Note that the information regarding the history of the action “a” presented to the user and the parameter of the value function is stored in the learned model storage unit 203.

Thus, the learned model can learn a strategy of presenting the optimum action “a” in the state s at the individual time of the user so that the user can go to bed at 24 o'clock. In addition, as illustrated in FIG. 6, the learned model corresponding to the agent schedules not only the specific action which is going to bed of the user but also the entire actions of the user so as to obtain the reward. Further, the learned model can guide the user to the healthy lifestyle habit by dynamically presenting the action “a” regarding which action is performed at the individual time.

Next, an operation of the information presentation device 10 will be explained.

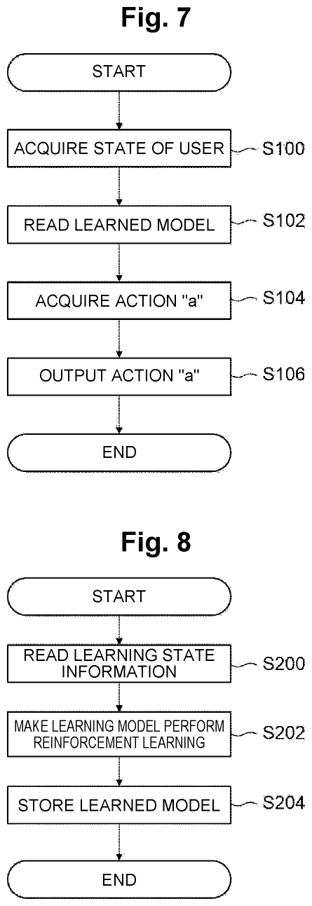

FIG. 7 is a flowchart illustrating a flow of information presentation processing by the information presentation device 10. The information presentation processing is performed by the CPU 11 reading the information presentation processing program stored in the ROM 12 or the storage 14, loading the program in the RAM 13 and executing the program.

The CPU 11 of the information presentation device 10 executes the information presentation processing illustrated in FIG. 7 when the state of the user input from the input unit 15 for example is received, as the state acquisition unit 101.

In step S100, the CPU 11 acquires the state of the user at the current time, as the state acquisition unit 101.

In step S102, the CPU 11 reads the learning model or the learned model stored in the learned model storage unit 203, as the action information acquisition unit 103.

In step S104, the CPU 11 inputs the state of the user at the current time acquired in step S200 described above to the learning model or the learned model read in step S102 described above, and acquires the action “a” that the user should take at the next time, as the action information acquisition unit 103.

In step S106, the CPU 11 outputs the action “a” acquired in step S104 described above, and ends the information presentation processing, as the information output unit 104.

The action “a” output from the information output unit 104 is displayed at the display unit 16, and the user takes the action according to the action “a”. In addition, the state acquisition unit 101 transmits the state of the user at the current time to the learning device 20.

Next, an operation of the learning device 20 will be explained.

FIG. 8 is a flowchart illustrating a flow of learning processing by the learning device 20. The learning processing is performed by the CPU 21 reading the learning program stored in the ROM 22 or the storage 24, loading the program in the RAM 23 and executing the program.

First, the CPU 21 acquires the state of the user at the current time transmitted from the information presentation device 10 and stores the state in the learning data storage unit 202 as the learning state, as the learning state acquisition unit 201. Then, the CPU 21 executes the learning processing illustrated in FIG. 8.

In step S200, the CPU 21 reads the learning state stored in the learning data storage unit 202, as the learning unit 204.

In step S202, the CPU 21 obtains a new learned model by subjecting the learning model or the learned model stored in the learned model storage unit 203 to the reinforcement learning so as to increase the total sum of the reward output from the preset reward function, based on the learning state read in step S200 described above, as the learning unit 204.

In step S204, the CPU 21 stores the new learned model obtained in step S202 described above in the learned model storage unit 203, as the learning unit 204.

By the execution of the learning processing described above, the parameter of the learning model or the learned model is updated, and the learned model for presenting the action according to the state of the user is stored in the learned model storage unit 203.

Note that, when the learned model is updated by the learning device 20 and the learned model is stored in the learned model storage unit 203 of the learning device 20, the learned model is stored in the learning model storage unit 102 of the information presentation device 10 via the communication means 30.

As explained above, the information presentation device 10 of the present embodiment inputs the state of the user to the learned model for outputting the action according to the state from the state of the user, the learned model being subjected to the reinforcement learning beforehand based on the reward function which outputs the reward according to the state of the user relative to the target state of the user. Then, the information presentation device 10 acquires the action according to the acquired state of the user, and outputs the acquired action. Thus, the action to be recommended can be presented in consideration of a chronological order of the actions of the user.

In addition, the learning device 20 of the present embodiment acquires a state of a user as a learning state, and subjects the learning model for outputting the action according to the state from the state of the user to the reinforcement learning based on the reward function which outputs the reward according to the learning state relative to the target state of the user, so as to increase the total sum of the reward output from the reward function. Then, the learning device 20 acquires the learned model which outputs the action according to the state of the user. Thus, the learned model capable of presenting the action to be recommended in consideration of the chronological order of the actions of the user can be obtained.

Further, the learning device 20 of the present embodiment can dynamically present the appropriate action in consideration of the entire daily actions of the user to the user.

Note that the information presentation processing and the learning processing executed by the CPU reading software (program) in the embodiment described above may be performed by various kinds of processors other than the CPU. Examples of the processors in this case are a PLD (Programmable Logic Device) for which a circuit configuration is changeable after manufacture such as an FPGA (Field-Programmable Gate Array) and an exclusive electric circuit which is a processor having the circuit configuration exclusively designed to execute specific processing such as an ASIC (Application Specific Integrated Circuit), etc. In addition, the information presentation processing and the learning processing may be executed by one of the various kinds of processors, or may be executed by a combination of two or more processors of a same kind or different kinds (for example, the plurality of FPGAs, and the combinations of the CPU and the FPGA, or the like). Further, a hardware structure of the various kinds of processors is, more specifically, an electric circuit for which circuit elements such as semiconductor elements are combined.

In addition, in the individual embodiment described above, the aspect in which the information presentation program is stored (installed) beforehand in the storage 14 and the learning program is stored (installed) beforehand in the storage 24 is explained, however, it is not limited thereto. The program may be provided in a form of being stored in a non-transitory storage medium such as a CD-ROM (Compact Disk Read Only Memory), a DVD-ROM (Digital Versatile Disk Read Only Memory), and a USB (Universal Serial Bus) memory. Further, the program may be in the form of being downloaded from an external device via a network.

Also, the information presentation processing and the learning processing of the present embodiment may be made up of a computer or a server or the like including a general-purpose arithmetic processing device and a storage device or the like, and each processing may be executed by the program. The program is stored in the storage device and can be recorded in a recording medium such as a magnetic disk, an optical disk and a semiconductor memory and also provided via a network. Needless to say, any other components are not necessarily needed to be implemented by a single computer or server, and may be implemented by being dispersed in a plurality of computers connected by a network.

The present embodiment is not limited to the individual embodiment described above, and various modifications and applications are possible without departing from the gist of the individual embodiment.

Regarding the above embodiment, the following supplementary notes are disclosed further.

(Supplementary Item 1)

An information presentation device including:

a memory; and

at least one processor connected to the memory,

wherein the processor is configured to

acquire a state of a user,

acquire an action according to the acquired state by inputting the acquired state to a learning model or a learned model for outputting the action according to the state from the state of the user, the learning model or the learned model being subjected to reinforcement learning based on a reward function which outputs a reward according to the state of the user relative to a target state of the user, and

output the acquired action.

(Supplementary Item 2)

A learning device including:

a memory; and

at least one processor connected to the memory,

wherein the processor is configured to

acquire a state of a user as a learning state, and

acquire a learned model which outputs an action according to the state of the user by subjecting a learning model to reinforcement learning, the learning model being for outputting the action according to the state from the state of the user, based on a reward function which outputs a reward according to the learning state relative to a target state of the user, so as to increase a total sum of the reward output from the reward function.

(Supplementary Item 3)

A non-transitory storage medium storing an information presentation program for making a computer execute processing of:

acquiring a state of a user;

acquiring an action according to the acquired state by inputting the acquired state to a learning model or a learned model for outputting the action according to the state from the state of the user, the learning model or the learned model being subjected to reinforcement learning based on a reward function which outputs a reward according to the state of the user relative to a target state of the user; and

outputting the acquired action.

(Supplementary Item 4)

A non-transitory storage medium storing a learning program for making a computer execute processing of:

acquiring a state of a user as a learning state; and

acquiring a learned model which outputs an action according to the state of the user by subjecting a learning model to reinforcement learning, the learning model being for outputting the action according to the state from the state of the user, based on a reward function which outputs a reward according to the learning state relative to a target state of the user, so as to increase a total sum of the reward output from the reward function.

### REFERENCE SIGNS LIST

10 Information presentation device
20 Learning device
101 State acquisition unit
102 Learning model storage unit
103 Action information acquisition unit
104 Information output unit
201 Learning state acquisition unit
202 Learning data storage unit
203 Learned model storage unit
204 Learning unit

1. An information presentation device comprising circuit configured to execute a method comprising:
acquiring a state of a user;
acquiring an action according to the state by inputting the state to either a learning model or a learned model for outputting the action according to the state from the state of the user, the learning model or the learned model being subjected to reinforcement learning based on a reward function which outputs a reward according to the state of the user relative to a target state of the user; and
outputting.

2. The information presentation device according to claim 1,
wherein the acquiring the state includes acquiring the state of the user at current time, and
wherein the reward function outputs the reward according to the state of the user at the current time relative to the target state of the user in the future.

3. The information presentation device according to claim 1,
wherein the reward function includes:
outputting a larger reward as the state of the user at the current time comes closer to the target state of the user in the future, and
outputting a smaller reward as the state of the user at the current time separates farther from the target state of the user in the future.

4. A learning device comprising circuit configured to execute a method comprising:
acquiring a state of a user as a learning state; and
acquiring a learned model, wherein the learned model outputs an action according to the state of the user by subjecting a learning model to reinforcement learning based on a reward function, and wherein the reward function outputs a reward according to the learning state relative to a target state of the user.

5. A computer-implemented method for presenting information associated with an action, the method comprising:
acquiring a state of a user;
acquiring an action according to the acquired state by inputting the acquired state to a learning model or a learned model for outputting the action according to the state from the state of the user, the learning model or the learned model being subjected to reinforcement learning based on a reward function which outputs a reward according to the state of the user relative to a target state of the user; and
outputting the acquired action.

6-8. (canceled)

9. The information presentation device according to claim 1, wherein the state includes observable information associated with at least one of time, a place, or a weather.

10. The information presentation device according to claim 1, wherein the state includes information associated with at least one of an action of the user or a health state of the user.

11. The information presentation device according to claim 1, wherein the reward function outputs a degree of the reward that increases as a difference between the state of the user at the current time comes and to the target state of the user becomes smaller.

12. The information presentation device according to claim 1, wherein the reinforcement learning uses a Markov decision process including:
determining a transition probability to a next state based on the action based on a set of states and a set of actions, and
determines the reward associated with the action.

13. The learning device according to claim 4, wherein the acquiring the state includes acquiring the state of the user at current time, and
wherein the reward function outputs the reward according to the state of the user at the current time relative to the target state of the user in the future.

14. The learning device according to claim 4, wherein the reward function includes:
outputting a larger reward as the state of the user at the current time comes closer to the target state of the user in the future, and
outputting a smaller reward as the state of the user at the current time separates farther from the target state of the user in the future.

15. The learning device according to claim 4, wherein the state includes observable information associated with at least one of time, a place, or a weather.

16. The learning device according to claim 4, wherein the state includes information associated with at least one of an action of the user or a health state of the user.

17. The learning device according to claim 4, wherein the reinforcement learning uses a Markov decision process including:
determining a transition probability to a next state based on the action based on a set of states and a set of actions, and
determines the reward associated with the action.

18. The learning device according to claim 4, wherein the reward function includes:
outputting a larger reward as the state of the user at the current time comes closer to the target state of the user in the future, and
outputting a smaller reward as the state of the user at the current time separates farther from the target state of the user in the future.

19. The computer-implemented method according to claim 5, wherein the acquiring the state includes acquiring the state of the user at current time, and
wherein the reward function outputs the reward according to the state of the user at the current time relative to the target state of the user in the future.

20. The computer-implemented method according to claim 5, wherein the reward function includes:
outputting a larger reward as the state of the user at the current time comes closer to the target state of the user in the future, and
outputting a smaller reward as the state of the user at the current time separates farther from the target state of the user in the future.

21. The computer-implemented method according to claim 5, wherein the state includes observable information associated with at least one of time, a place, or a weather, and
wherein the state includes information associated with at least one of an action of the user or a health state of the user.

22. The computer-implemented method according to claim 5, wherein the reinforcement learning uses a Markov decision process including:
determining a transition probability to a next state based on the action based on a set of states and a set of actions, and
determines the reward associated with the action.

23. The computer-implemented method according to claim 5, wherein the reward function includes:
outputting a larger reward as the state of the user at the current time comes closer to the target state of the user in the future, and
outputting a smaller reward as the state of the user at the current time separates farther from the target state of the user in the future.
